(19) United States
(12) Patent Application Publication
(10) Pub. No.: US 2023/0392109 A1
(43) Pub. Date: Dec. 7, 2023

(54) APPARATUS AND METHODS FOR LIQUID CULTURE PRODUCTION AND INOCULATION

(71) Applicant: BioReactive Technologies Inc., Vancouver (CA)

(72) Inventors: Michael James MCNULTY, Roseville, CA (US); Nathan Parker FRY, Roseville, CA (US); Evin Marie REVELLO, Roseville, CA (US)

(21) Appl. No.: 18/203,526

(22) Filed: May 30, 2023

Related U.S. Application Data
(60) Provisional application No. 63/348,401, filed on Jun. 2, 2022.

(51) Int. Cl. C12N 1/14; A01G 18/50; C12M 1/04; C12M 1/00; C12M 1/12

(52) U.S. Cl. CPC C12N 1/14; A01G 18/50; C12M 23/24; C12M 29/08; C12M 23/14; C12M 25/14; A01G 18/20

(57) ABSTRACT

An inoculation apparatus is provided. The inoculation apparatus includes a vessel for containing liquid mycelium culture having: an outlet port; an injection port; and a breather port. The inoculation apparatus also includes an auto-fill syringe having a nozzle and an inlet port; a connection line with a first end connecting to the outlet port and a second end connecting to the inlet port; and an injection means connected to the nozzle. Related methods are also provided.

### CROSS-REFERENCE TO RELATED APPLICATIONS

This application claims priority from, and for the purpose of the United States of America the benefit under 35 U.S.C. § 119 in connection with, U.S. application No. 63/348,401 filed 2 Jun. 2022 and entitled APPARATUS AND METHODS FOR LIQUID CULTURE PRODUCTION AND INOCULATION which is hereby incorporated herein by reference for all purposes.

### TECHNICAL FIELD

The present invention relates to an apparatus and methods for inoculating liquid culture, for example for spawn production.

### BACKGROUND

Cultivation of fungi, and in particular mushrooms, may include a step of culturing mycelium in a liquid nutrient broth or on solid agar. In liquid culture, once the mycelium has sufficiently expanded, the mycelium rich suspension may be inoculated into spawn bags, i.e., sealed bags containing sterilized grains. The grains provide a nutrient surface upon which the mycelium can further colonize before being introduced to a substrate where the mushroom is ultimately grown.

Single use needle syringes are typically used to inject the mycelial liquid culture into spawn bags. When inoculating multiple spawn bags in succession, contamination may occur each time the syringe is refilled. Refilling the syringe also takes time. While larger syringes may be used, these may be awkward to handle and settling of the mycelial suspension can occur within the syringe, resulting in uneven rates of inoculation. Further, maintaining a desired temperature of mycelial liquid culture may be hampered by the constant refilling, the shape, and the limited volume capacity of single use syringes.

Improved apparatus and methods for producing and inoculating liquid culture are desirable.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides an inoculation apparatus comprising: a vessel for containing liquid mycelium culture comprising: an outlet port; an injection port; and a breather port; an auto-fill syringe comprising a nozzle and an inlet port; a connection line with a first end connecting to the outlet port and a second end connecting to the inlet port; and an injection means connected to the nozzle.

The first end of the connection line may comprise a female keg fitting and the outlet port may comprise a male keg fitting with a stem extending into the vessel, the male keg fitting connected to the female keg fitting. The injection port may comprise an aperture covered by a self-healing elastomer. The injection means may comprise a needle. The needle may comprise a sealed end and a plurality of radially arranged holes. The injection means may comprise a second nozzle comprising a distal face having plurality of angled holes. The vessel may comprise a draft column and aeration means disposed in the draft column. An air pump may be connected to the vessel and in fluid communication with the aeration means. A temperature regulating means connected between the air pump and the aeration means may be provided. The temperature regulating means may comprise an inline heater. The vessel may comprise a detachable lid, the detachable lid comprising the outlet port, the injection port and the breather port. The vessel may comprise a blender attachment lid interchangeable with the detachable lid, the blender attachment lid comprising a rotatable blade assembly, wherein the blender attachment lid is coupleable to a blender rotary drive for actuating the rotatable blade assembly.

Another aspect of the invention provides a method of inoculating liquid culture for spawn production. The method comprises: a) providing an inoculation apparatus as described herein; b) injecting mycelial liquid culture into the vessel; c) growing the mycelial liquid culture to a desired maturity; d) providing a plurality of spawn containers containing sterilized grains; e) serially inoculating the plurality of spawn containers by injecting the mycelial liquid culture on to the sterilized grains in the spawn container.

The spawn container may comprise a spawn bag and the injection means may comprise a needle, and wherein step e) may comprise inoculating the spawn bag with a dose of the mycelium liquid culture equivalent to multiple volumes of the auto-fill syringe without withdrawing the needle from an injection port of the spawn bag by allowing auto-filling of the syringe with the mycelium culture from the vessel between injections.

The injection means may comprise a second nozzle comprising a plurality of holes and wherein step e) comprises inoculating the sterilized grains with simultaneous multiple streams of mycelium liquid culture.

Step c) may comprise providing temperature regulated aeration to the culture.

After step c) may comprise exchanging the lid of the vessel with a lid with a blender attachment lid and blending the culture into a slurry.

Step e) may comprise inoculating at a rate of 20 to 60 mL of the mycelium liquid culture per pound of the sterilized grains.

The grain may be selected from the group consisting of rye, barley, rice, millet, wheat, and popcorn.

Another aspect of the invention provides a method of inoculating plant roots, the method comprising: a) providing an inoculation apparatus as described herein, wherein the vessel contains rhizobial liquid culture; and b) serially inoculating a plurality of plants by injecting the rhyziobial liquid culture adjacent the plant roots.

In addition to the exemplary aspects and embodiments described above, further aspects and embodiments will become apparent by reference to the drawings and by study of the following detailed descriptions.

### BRIEF DESCRIPTION OF THE DRAWINGS

In drawings which illustrate non-limiting embodiments of the invention:

FIG. 1 shows an inoculation apparatus according to an embodiment of the invention.

FIG. 2 shows a vessel assembly of the inoculation apparatus of FIG. 1.

FIG. 3 shows an injection assembly of the inoculation apparatus of FIG. 1.

FIG. 4 shows a vessel assembly of an inoculation apparatus according to an embodiment of the invention.

FIG. 5 shows the inoculation apparatus of FIG. 1 with multiple spawn bags.

FIG. 6A (end perspective view), 6B (side elevation cross-sectional view) and 6C (end view) show an alternative nozzle for attachment to an injection assembly of the inoculation apparatus of FIG. 1.

FIG. 6D shows an alternative needle for attachment to an injection assembly of the inoculation apparatus of FIG. 1.

FIGS. 7A and 7B show a vessel assembly according to another embodiment of the invention.

FIG. 8 shows the vessel assembly of FIG. 1 with an air pump, inline heater, and inline filter.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

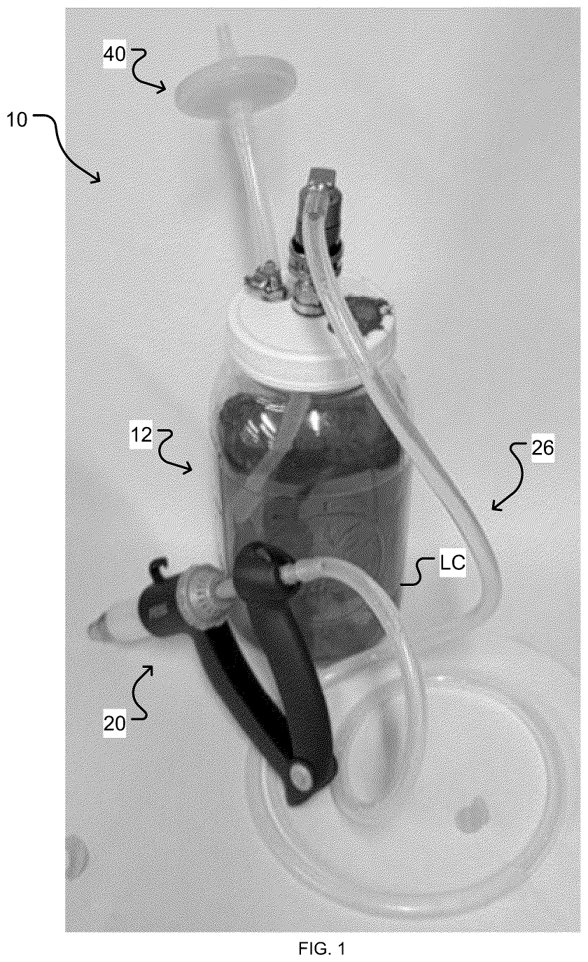

FIGS. 1 to 3 and 5 show an inoculation apparatus 10 according to an embodiment. Inoculation apparatus 10 generally comprises a vessel assembly 11 connected to an injection assembly 19.

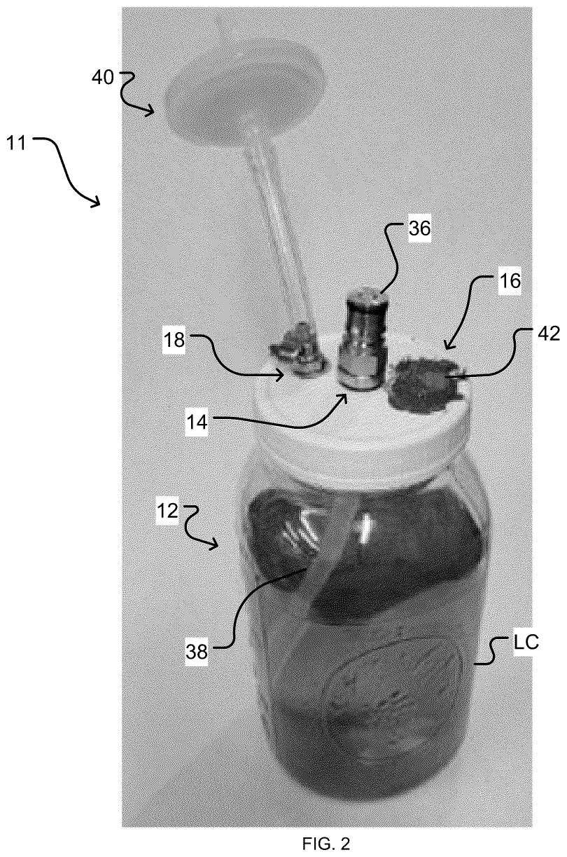

As best shown in FIG. 2, vessel assembly 11 includes a vessel 12 with a sterile interior for holding liquid culture LC. Vessel 12 is shown as a glass jar in the illustrated embodiment but may be any other suitable container that can be sterilized. Vessel 12 includes an outlet port 14 through which liquid culture LC can be withdrawn, an injection port 16 through which an initial culture is injected, and a breather port 18 through which liquid culture LC can receive oxygen. Outlet port 14 may be fitted with a male keg fitting 36. Outlet port 14 is also in fluid communication with a down stem 38 that extending into liquid culture LC. Injection port 16 is comprised of a self-healing elastomer, such as silicon caulk, which provides sealed access to the interior of vessel 10. Breather port 18 is provided with a filter 40, such as a 0.2 micron filter, to maintain sterility of the interior of vessel 10 while allowing gas exchange between the interior of vessel 10 and the outer environment of vessel 10.

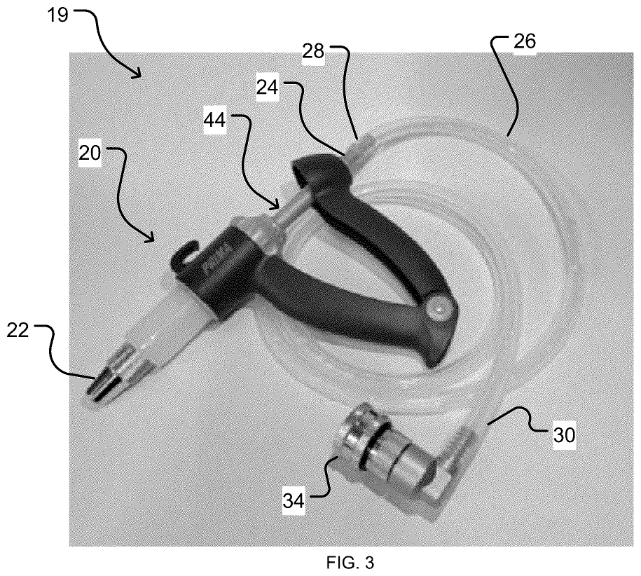

As best shown in FIG. 3, injector assembly 19 includes an auto-fill syringe 20 with a spring tensioned plunger 44. An inlet port 24 of auto-fill syringe 20 is connected to a first end 28 of a flexible fluid connection line 26. A second end 30 of connection line 26 is connected to a female keg fitting 34. Female keg fitting 34 of injector assembly 19 mates with male keg fitting 36 of vessel assembly 11. Syringe 20 also has a nozzle 22 onto which a disposable needle 32 may be fitted.

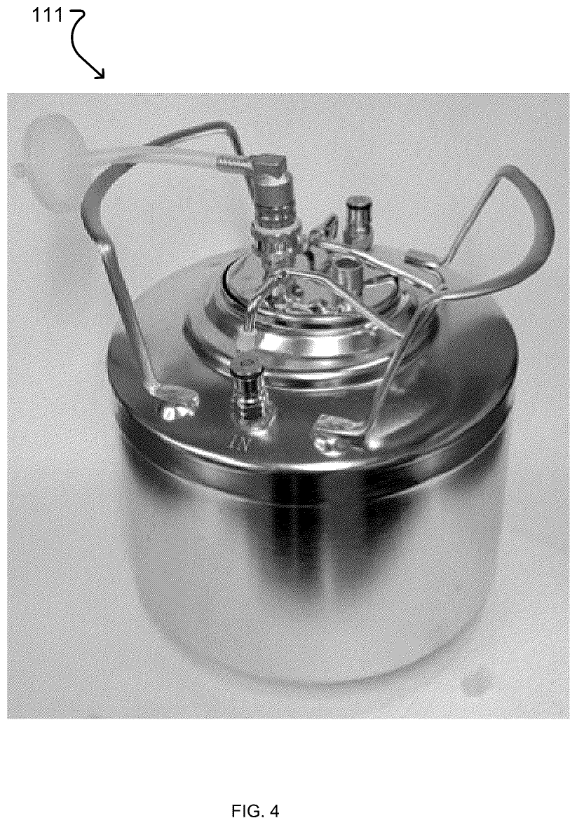

FIG. 4 shows a larger vessel assembly 111 according to another embodiment, for culturing and storing larger volumes of liquid culture LC. Vessel assembly 111 may for example substitute for vessel assembly 11 in apparatus 10.

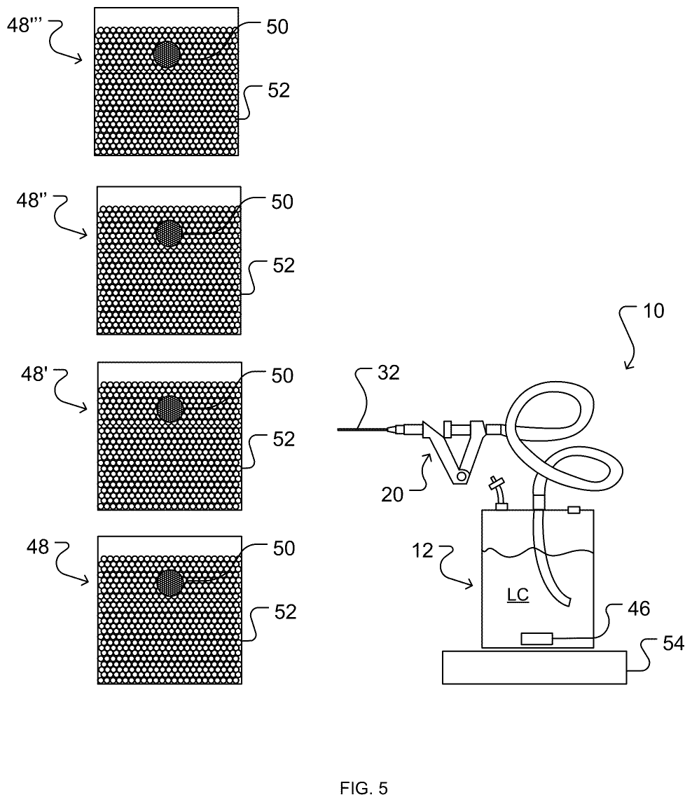

FIG. 5 shows inoculation apparatus 10 and several spawn bags 48, 48′, 48″, 48′″. Each spawn bag 48, 48′, 48″, 48′″ has an injection port 50 through which liquid culture LC is injected by inoculation apparatus 10. Each spawn bag 48, 48′, 48″, 48′″ has a sterile interior and is filled with sterilized grains 52, such as rye, barley, rice, millet, wheat, and/or popcorn.

Inoculation apparatus 10 allows for serial inoculation of several spawn bags in rapid succession without manual refiling of a syringe after each injection and/or between spawn bags. In particular a user can inoculate a first spawn bag 48 by piercing its injection port with needle 32 of injection assembly 19 of inoculation apparatus 10. Mycelial liquid culture LC is injected into first spawn bag 48. Multiple loads of auto-fill syringe 20 can be effected by the user by manually “pumping” the syringe (here, squeezing the V-shaped handle) multiple times to allow a greater volume of liquid culture LC to be injected into spawn bag 48, as well as to allow subsequent injection of spawn bags 48′, 48″ and 48′″ in rapid succession. In some embodiments, 5 to 100 mL, or 10 to 80 mL, or 15 to 70 mL, or 20 to 60 mL of mycelial liquid culture LC may be injected per pound of sterilized grains 48. In some embodiments grains 48 may be provided in a spawn container other than a spawn bag, such as sterile jars and the like.

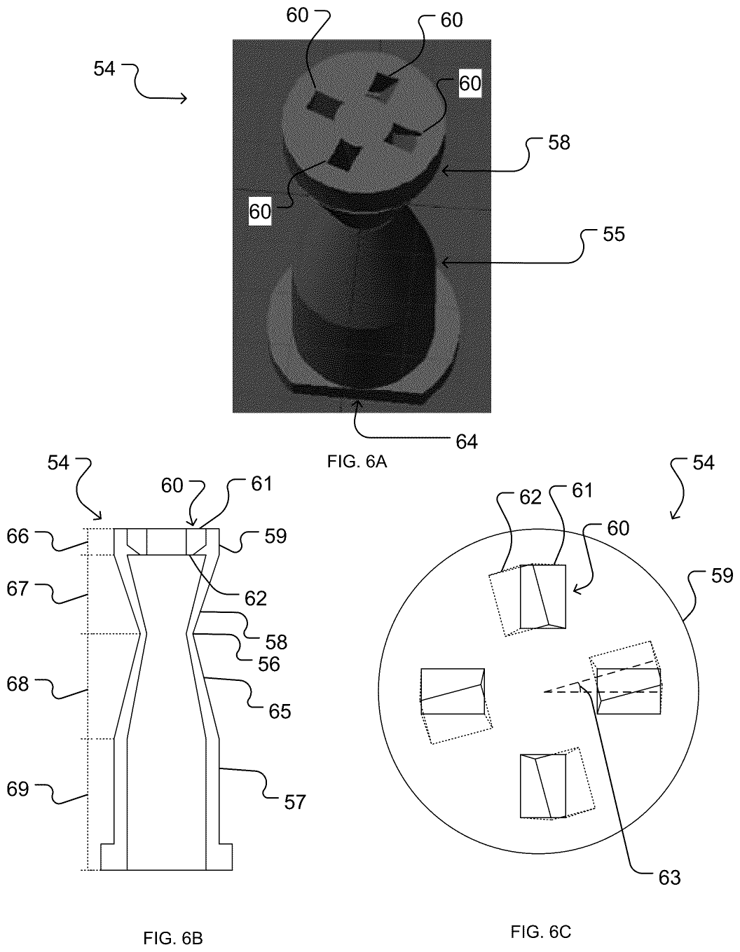

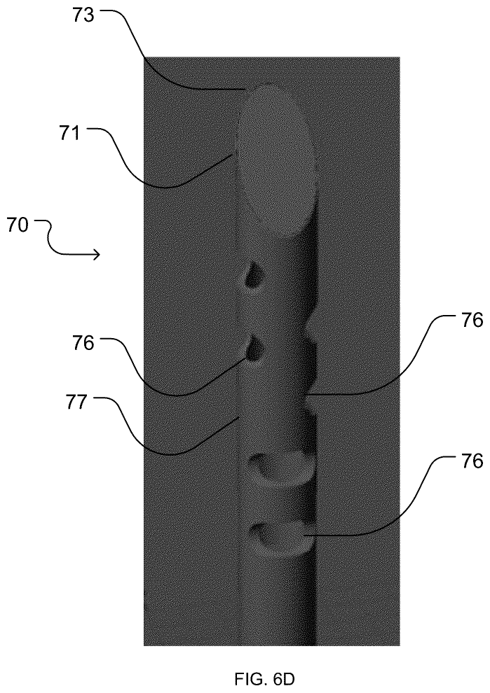

FIGS. 6A to 6C show a nozzle 54 that may be used as an alternative to needle 32 in some embodiments of injection assembly 19. Nozzle 54 has a body 55 having a generally double napped cone shaped vertical cross-section defined by a narrowing portion 65 and widening portion 58. Body 55 also includes a base 57. Widening portion 58 has a distal wall 59 with a plurality of holes 60 extending through distal wall 59. In some embodiments, distal wall 59 may have 2, 3, 4, 5, or 6 holes. Holes 60 are defined by an outer opening 61 and an inner opening 62. In some embodiments holes 60 are radially arranged on circular distal wall 59. In some embodiments outer openings 61 and inner openings 62 are shifted, either clockwise or counter-clockwise, with respect to each other, such that the holes 60 are angular. In some embodiments, an angle 63 of this shift may be about 5 to 25 degrees, or about 10 to 20 degrees, or about 15 degrees. In some embodiments the ratio of a thickness 66 of distal wall 59 and a height 67 of widening portion 58 may be about 1:3. In some embodiments the ratio of a thickness 66 of distal wall 59, a height 67 of widening portion 58, and a height 68 of narrowing portion 65 may be about 1:3:4. In some embodiments the ratio of a thickness 66 of distal wall 59, a height 67 of widening portion 58, a height 68 of narrowing portion 65, and a height 69 of base 57 may be about 1:3:4:5. In some embodiment, holes 60 may have a diameter or width ranging from about 200 um to 1.5 mm, or about 500 um to 1.2 mm, or about 700 um to 1 mm.

Body 55 includes an attachment means 64 for attaching to auto-fill syringe 20. Attachment means 64 may for example be a luer lock fitting. In the case of a spawn bag 48, instead of piercing with a needle, spawn bag 48 may be opened in a sterile environment and nozzle 54 aimed to provide a split stream of liquid culture LC to sterilized grains 48. In the case of jar inoculation, the jar may be opened in a sterile environment and nozzle 54 aimed to provide a split stream of liquid culture LC to sterilized grains in the jar. The double napped cone shape of body 55 and angled holes 60 of nozzle 54 work together to guide liquid culture LC into multiple streams from nozzle 54 that substantially spread from a horizontal to vertical when viewed from the side, and substantially 360 degrees around when viewed end-on.

FIG. 6D shows a needle 70 that may be used as an alternative to needle 32 in some embodiments of injection assembly 19. Needle 70 has a plugged end 71 with sharp tip 73 and a plurality of radial holes 76 along its barrel 77. In some embodiments, a standard needle may be soldered closed, for example with silver, to form plugged end 71, and slots may be cut out to form holes 76. In other embodiments needle 70 may be initially constructed in its final form. In some embodiments, radial holes 76 may be equidistantly arranged to provide 2, 3, 4, 5, 6, 7, 8, 9 or 10 radially equidistant streams normal to the long axis of needle 70. In some embodiments, pairs of radial holes 76 aligned along the long axis of needle 70 may be staggeringly and evenly arranged around needle 70, as shown in FIG. 6C.

The multiple streams created by nozzle 54 and needle 70 create multiple inoculation sites in grains 48 for more effective inoculation.

In some embodiments, before and/or during inoculation, vessel 12 of inoculation apparatus 10 may be placed on a magnetic stirrer 54, with sterilized stir bar 46 inside vessel 12 to mix mycelial liquid culture LC to ensure a consistent amount of mycelium is being inoculated per load of auto-fill syringe 20. In some embodiments, where a particular temperature of mycelial liquid culture LC is desirable during inoculation, vessel 12 may be kept warmer by suitable means such as warming plate (not shown) or may be kept cooler by suitable means such as at least partially submersion in a cooler (not shown), to maintain the desired temperature of mycelial liquid culture LC throughout the inoculation process.

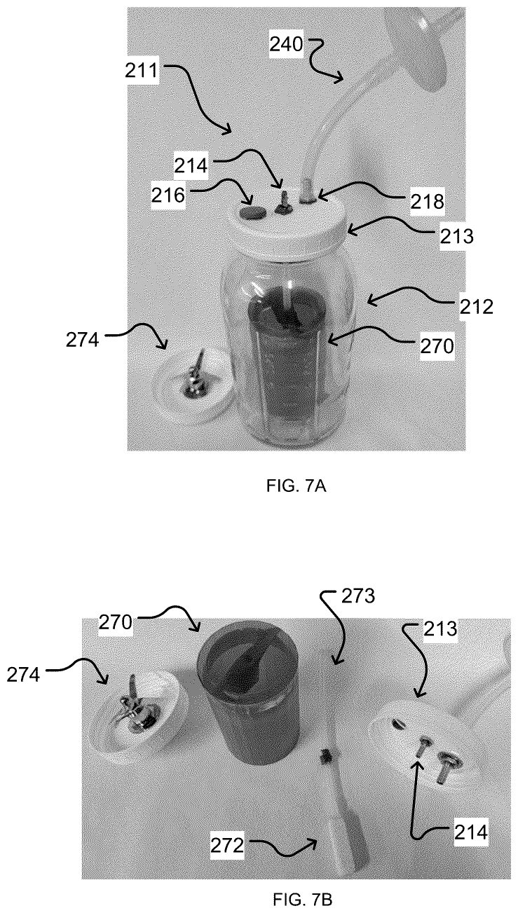

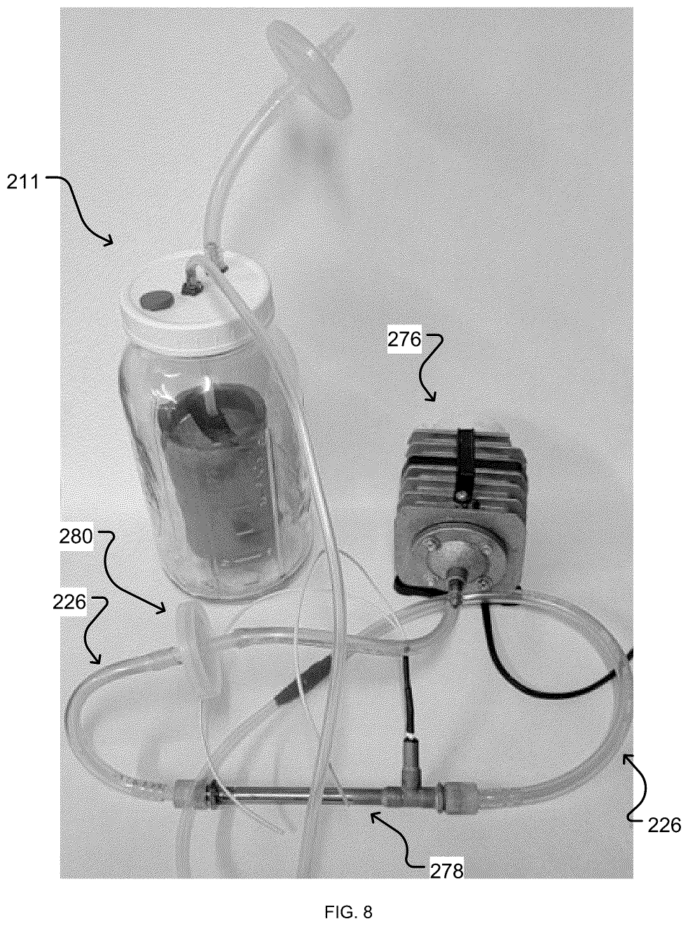

FIGS. 7A, 7B and 8 show a vessel assembly 211 according to another embodiment. Vessel assembly 211 may for example substitute for vessel assembly 11 in apparatus 10. Vessel assembly 211 is an internal loop or airlift bioreactor and includes a vessel 212 with a lid 213 that has a inlet/outlet port 214, an injection port 216 and a breather port 218. Inlet/outlet port 213 may connect to a tubing 273 for an aeration means 272 inside a draft column 270 within vessel 212. Aeration means 272 may for example be an aeration stone, such as a 10 um glass aeration stone. Air is supplied to aeration means by an air pump 276. In some embodiments air from air pump 276 is filtered by an inline air filter 280 (such as a 0.2 um filter) and/or heated by an inline heater 278 before being bubbled into draft column 270 by aeration means 272. In some embodiments, depending on the desired culturing temperature, inline heater 278 may be substituted with an inline cooler.

Once liquid culture LC is sufficiently mature, draft column 270, tubing 273 and aeration means 272 and lid 213 may be replaced with a sterilized blender attachment lid 274. Blender attachment lid 274 includes a rotatable blade assembly. Blender attachment lid 274 is connected to a blender rotary drive (not shown) and mycelial pellets produced in vessel 212 are blended into a liquid culture LC slurry. Blending the liquid culture LC into a slurry reduces the occurrence of clogging of injector assembly 19 during inoculation. The blender attachment is then replaced with lid 213 and the inlet/outlet port is suitably connected by connection line 26 to an injection assembly 19. The foregoing steps would typically be conducted in a sterile environment, e.g. in a laminar flow cabinet or the like.

In some embodiments, inoculation apparatus 10 allows for serial inoculation of rhizobial liquid culture for plant roots in a similar manner as described herein for mycelial liquid culture for spawn bags.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1. An inoculation apparatus comprising:
a vessel for containing liquid mycelium culture comprising:
an outlet port;
an injection port; and
a breather port;
an auto-fill syringe comprising a nozzle and an inlet port;
a connection line with a first end connecting to the outlet port and a second end connecting to the inlet port; and
an injection means connected to the nozzle.

2. An inoculation apparatus according to claim 1 wherein the first end of the connection line comprises a female keg fitting and the outlet port comprises a male keg fitting with a stem extending into the vessel, the male keg fitting connected to the female keg fitting.

3. An inoculation apparatus according to claim 1 wherein the injection port comprises an aperture covered by a self-healing elastomer.

4. An inoculation apparatus according to claim 1 wherein the injection means comprises a needle.

5. An inoculation apparatus according to claim 4 wherein the needle comprises a sealed end and a plurality of radially arranged holes.

6. An inoculation apparatus according to claim 1 wherein the injection means comprises a second nozzle comprising a distal face having plurality of angled holes.

7. An inoculation apparatus according to claim 1 wherein the vessel comprises a draft column and aeration means disposed in the draft column.

8. An inoculation apparatus according to claim 7 further comprising an air pump connected to the vessel and in fluid communication with the aeration means.

9. An inoculation apparatus according to claim 8 further comprising a temperature regulating means connected between the air pump and the aeration means.

10. An inoculation apparatus according to claim 9 wherein the temperature regulating means comprises an inline heater.

11. An inoculation apparatus according to claim 1 wherein the vessel comprises detachable lid, the detachable lid comprising the outlet port, the injection port and the breather port.

12. An inoculation apparatus according to claim 11 wherein the vessel comprises a blender attachment lid interchangeable with the detachable lid, the blender attachment lid comprising a rotatable blade assembly, wherein the blender attachment lid is coupleable to a blender rotary drive for actuating the rotatable blade assembly.

13. A method of inoculating liquid culture for spawn production, the method comprising:
a) providing an inoculation apparatus according to claim 1;
b) injecting mycelial liquid culture into the vessel;
c) growing the mycelial liquid culture to a desired maturity;
d) providing a plurality of spawn containers containing sterilized grains;
e) serially inoculating the plurality of spawn containers by injecting the mycelial liquid culture on to the sterilized grains in the spawn container.

14. A method according to claim 13 wherein the spawn container comprises a spawn bag and the injection means comprises a needle, and wherein step e) comprises inoculating the spawn bag with a dose of the mycelium liquid culture equivalent to multiple volumes of the auto-fill syringe without withdrawing the needle from an injection port of the spawn bag by allowing auto-filling of the syringe with the mycelium culture from the vessel between injections.

15. A method according to claim 13 wherein the injection means comprises a second nozzle comprising a plurality of holes and wherein step e) comprises inoculating the sterilized grains with simultaneous multiple streams of mycelium liquid culture.

16. A method according to claim 13 wherein step c) comprises providing temperature regulated aeration to the culture.

17. A method according to claim 13 wherein after step c) exchanging the lid of the vessel with a lid with a blender attachment lid and blending the culture into a slurry.

18. A method according to claim 13 wherein step e) comprises inoculating at a rate of 20 to 60 mL of the mycelium liquid culture per pound of the sterilized grains.

19. A method according to claim 13 wherein the grain is selected from the group consisting of rye, barley, rice, millet, wheat, and popcorn.

20. A method of inoculating plant roots, the method comprising:
a) providing an inoculation apparatus according to claim 1 wherein the vessel contains rhizobial liquid culture; and
b) serially inoculating a plurality of plants by injecting the rhyziobial liquid culture adjacent the plant roots.
